(19) United States
(12) Plant Patent Application Publication
(10) Pub. No.: US 2015/0135376 P1
(43) Pub. Date: May 14, 2015

(54) Hop Plant Named 'HBC 564'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 13/998,587

(22) Filed: Nov. 13, 2013

(51) Int. Cl. A01H 5/00

(52) U.S. Cl. PLT/236

(57) ABSTRACT

A new hop plant named ‘HBC 564’ is disclosed. The cones of ‘HBC 564’ mature in mid-September, and yield a crop of 2200 to 2600 pounds per acre. ‘HBC 564’ is used for its very unique aromatic quality, high alpha acid content and exceptional yield.

### CROSS-REFERENCE TO RELATED APPLICATIONS

None

### STATEMENT REGARDING FEDERALLY SPONSORED RESEARCH OR DEVELOPMENT

None

### GENUS AND SPECIES

Humulus lupulus

### VARIETY DENOMINATION

‘HBC 564’

### BACKGROUND OF THE INVENTION

‘HBC 564’ is a product of a controlled breeding program carried out by the inventors in the Yakima Valley of Washington state. ‘HBC 564’ was one of several seedlings resulting from a cross made in 2007 of female parent ‘Tillicum’ (patented) and male parent ‘HBC 20-9-10’ (not patented). A single plant of ‘HBC 564’ was discovered in 2010 and used in a brewing trial during the winter of 2010-2011. Based on the preliminary brewing trial, ‘HBC 564’ was expanded in 2011 to one acre in the Toppenish, Wash. area to test large scale growing, brewing and solvent extraction beginning with the 2012 crop. Through several generations of asexual propagation by softwood cuttings at a greenhouse facility in Yakima, Wash. ‘HBC 564’ has been observed to retain its distinctive characteristics and remain true to type.

### BRIEF DESCRIPTION OF THE PHOTOGRAPH

FIG. 1 illustrates the dried and pressed hop cones of ‘HBC 564’.

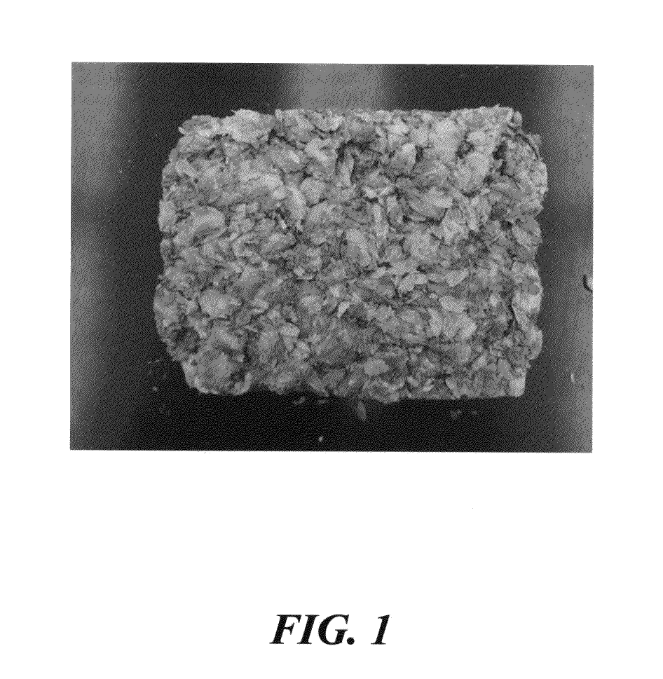

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2012 growing season at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average.
Ploidy: Triploid
Use: Brewing
Disease susceptibility: ‘HBC 564’ is not resistant to infestations of hop downy mildew incited by Pseudoperonospora humuli. ‘HBC 564’ is resistant to strains of hop powdery mildew found in the Yakima Valley and incited by Podosphaera macularis.
Pest susceptibility: ‘HBC 564’ is not resistant to normal infestations of hop aphid Phorodon humuli and two-spotted spider mite Tetranychus urticae.
Harvest date: September 9-15 (2012 growing season, Toppenish, Wash.)
Crop yield: 2200 to 2600 pounds per acre
Analytical characteristics:
   Alpha acid.—15% to 16%.
   Beta acid.—7.5% to 8.5%.
   Cohumulone.—32% to 34%.
   Total oil.—1.8 to 2.3 mL per 100 g cones.
   Storage stability.—70% to 80% alpha acids remaining after six months storage at room temperature.

1. What is claimed is a new and distinct hop plant as shown and described herein.
